(19) United States
(12) Patent Application Publication
(10) Pub. No.: US 2008/0242726 A1
(43) Pub. Date: Oct. 2, 2008

(54) Trans-Clomiphene for the Treatment of Benign Prostate Hypertrophy, Porstate Cancer, Hypogonadism Elevated Triglycerides and High Cholesterol

(76) Inventors: Joseph S. Podolski, The Woodlands, TX (US); Ronald D. Wiehle, Houston, TX (US)

Correspondence Address: HOWREY LLP - DC, C/O IP DOCKETING DEPARTMENT, 2941 FAIRVIEW PARK DR, SUITE 200, FALLS CHURCH, VA, 22042-2924, US

(21) Appl. No.: 11/571,150

(22) PCT Filed: Jul. 14, 2005
(86) PCT No.: PCT/US05/25000

§ 371 Date: Jan. 7, 2008

Related U.S. Application Data
(60) Provisional application No. 60/588,130, filed on Jul. 14, 2004.
(60) Provisional application No. 60/588,223, filed on Jul. 14, 2004.
(60) Provisional application No. 60/588,123, filed on Jul. 14, 2004.

(51) Int. Cl. A61K 31/135; A61P 43/00; A61P 35/00

(52) U.S. Cl. 514/551

(57) ABSTRACT

Compositions comprising trans-clomiphene may be used in treating benign prostate hypertrophy, prostate cancer, elevated triglyceride levels and hypogonadism.

### CROSS REFERENCE TO RELATED APPLICATIONS

The present application claims the benefit of U.S. Provisional Application No. 60/588,123 filed Jul. 14, 2004, U.S. Provisional Application No. 60/588,223 filed Jul. 14, 2004, and U.S. Provisional Application No. 60/588,130 filed Jul. 14, 2004, each of which is incorporated herein in its entirety.

### FIELD OF THE INVENTION

The present invention relates to the use of a composition comprising clomiphene.

### BACKGROUND

Testosterone is the primary male androgen, playing a vital role in overall male health. Testosterone is essential to the development and maintenance of specific reproductive tissues (testes, prostate, epididymis, seminal vesicle, and penis) and male secondary sex characteristics. It plays a key role in libido and erectile function and is necessary for the initiation and maintenance of spermatogenesis. Testosterone also has important functions not related to reproductive tissues. For example, it positively affects body composition by increasing nitrogen retention, which supports lean body mass, muscle size and strength. It also acts on bone to stimulate bone formation.

Testosterone secretion is the end product of a series of hormonal processes. Gonadotropin-releasing hormone (GnRH), which is secreted in the hypothalamus, controls the pulsatile secretion of luteinizing hormone (LH) and follicle stimulating hormone (FSH), which are secreted by the anterior pituitary. LH, in turn, regulates the production and secretion of testosterone in the Leydig cells of the testes, while FSH assists in inducing spermatogenesis. Testosterone is most often measured as “total testosterone.” This measurement includes testosterone that is bound to sex hormone-binding globulin (SHBG) (˜44%) and is therefore not bioavailable and testosterone which either is free (˜2%) or loosely bound to other proteins (non-SHBG-bound) (˜54%).

Results from a WHO study indicate that testosterone is normally secreted in a circadian rhythm, with higher levels in the morning and nadir levels occurring around 8 to 10 p.m. See FIG. 1. This variation in testosterone secretion throughout the day becomes much less pronounced in older men (mean age equals 71 years). The importance of this rhythm is not known at this time. Samples were obtained from both young and elderly patients every 10 minutes for 24 hours via an indwelling cannula. According to Tenover (1987) the mean 24 hr total serum testosterone levels in healthy young men (age range 22 yrs.-35 yrs. mean 27.3 yrs) was 4.9±0.3 (±SEM) mg/ml (17.0 nmol/L) while older men (age range 65 yrs-84 yrs. mean 70.7 yrs.) had a significantly lower mean 24 hrs. total serum testosterone level of 4.1±0.4 mg/ml. (P<0.5; 14.2 nmol/L). Total serum testosterone levels obtained from single random samples were also significantly lower in older men (4.0±0.2 mg/ml [13.9 nmol/L]) as compared to 4.8±0.2 mg/ml [16.6 nmol/L] in healthy young men.

Testosterone deficiency can result from underlying disease or genetic disorders and is also frequently a complication of aging. For example, primary hypogonadism results from primary testicular failure. In this situation, testosterone levels are low and levels of pituitary gonadotropins (LH and FSH) are elevated. Secondary hypogonadism is due to inadequate secretion of the pituitary gonadotropins. In addition to a low testosterone level, LH and FSH levels are low or low-normal. Some of the sequelae of adult testosterone deficiency include a wide variety of symptoms including: loss of libido, erectile dysfunction, oligospermia or azoospermia, absence or regression of secondary sexual characteristics, progressive decrease in muscle mass, fatigue, depressed mood and increased risk of osteoporosis. Many of these disorders are generically referred to as male menopause.

Several forms of testosterone therapy exist in the United States today. Recently, transdermal preparations have gained favor in the market. However, a scrotal testosterone patch results in supraphysiologic levels of 5α-dihydrotestosterone (DHT) due to the high concentration of 5α-reductase in scrotal skin. Nonscrotal systems are considered more convenient and most patients achieve average serum concentrations within the normal range and have normal levels of DHT. Oral testosterone therapy is not recommended because doses required for replacement therapy are associated with significant risk of hepatotoxicity.

### SUMMARY

The present invention is related to methods and compositions comprising trans-clomiphene. The composition may also comprise cis-clomiphene. The composition may comprise greater than 1/1 w/w of trans-clomiphene. The composition may comprise 0% to 29% weight/weight of (cis, -Z-, trans-clomiphene) (“cis-clomiphene” or “Zuclomid”) and 100% to 71% w/w (trans-, E-, cis-clomiphene) (“trans-clomiphene” or “Enclomid”) or pharmaceutically acceptable salts or solvates thereof. The composition may contain both cis-clomiphene and trans-clomiphene, and the ratio of trans-clomiphene and cis-clomiphene may be greater than 71/29. The composition may further comprise suitable pharmaceutical excipients, diluents, carriers, and the like. The composition may also comprise analogs of cis-clomiphene and trans-clomiphene isomers.

The composition may be formulated for a variety of methods of administration including oral, intravenous, subcutaneous, buccal, transmucosal, intrathecal, intradermal, and intracisternal administration. The composition may also be formulated for extended release. In some methods, the compositions may be administered so as to give rise to serum testosterone levels generally similar to the serum testosterone levels of a normal patient. Dosages of the composition may be administered in a pharmaceutical formulation that would give rise to peak serum testosterone levels between about 4 a.m. and noon. The composition may also be administered to give rise to peak serum testosterone levels at around 8 a.m. Alternatively, the composition may be administered to give rise to peak serum testosterone levels at any time of day deemed suitable by the patient and/or the prescribing physician.

The composition may be used for treating conditions such as prostate cancer and benign prostate hypertrophy. In treating prostate cancer, the composition may be administered in a treatment regimen also including a chemotherapeutic. Exemplary chemotherapeutics include Taxotere® (docetaxel), Novantrone® (mitoxantrone hydrochloride), Emcyt® (estramustine sodium phosphate) doxorubicin, ketoconazole and vinblastine although other chemotherapeutics may also be used. In some cases, the chemotherapeutic may be administered closely in time to the dosage of the composition. In other cases the chemotherapeutic may be administered to the patient during the same period of time in which the patient is undergoing treatment with the composition. Treatment with the composition may precede or follow treatment with the chemotherapeutic.

The composition may also be used for increasing serum testosterone levels in hypogonadal male mammals, and for ameliorating or preventing the sequelae of low testosterone levels. Some symptoms, or sequelae, of low testosterone levels, which the present invention may be used to treat, include reduction of muscle mass, limitation of body performance capacity, reduction of bone density, reduction of libido, and/or reduction of potency.

The composition may also be used for decreasing triglyceride levels in mammals, and more specifically in male mammals, and for ameliorating or preventing the sequelae of high cholesterol levels. The composition may also be used in treating high cholesterol levels in mammals, and more specifically in male mammals. Although methods of decreasing triglyceride levels and ameliorating or preventing the sequelae of high cholesterol levels are preferably used to treat males, they may also be used to treat females.

### BRIEF DESCRIPTION OF THE DRAWING

FIG. 1 is a graphic representative of the normal secretory total serum testosterone profiles in healthy men (young and old).

FIG. 2 shows the chemical structure of clomiphene citrate.

FIG. 3 is a graphic demonstration of the time course of serum testosterone levels with Clomid, Enclomid and Zuclomid.

FIG. 4 is a graphic demonstration of the time course of cholesterol levels in baboon males treated with Clomid, Enclomid and Zuclomid.

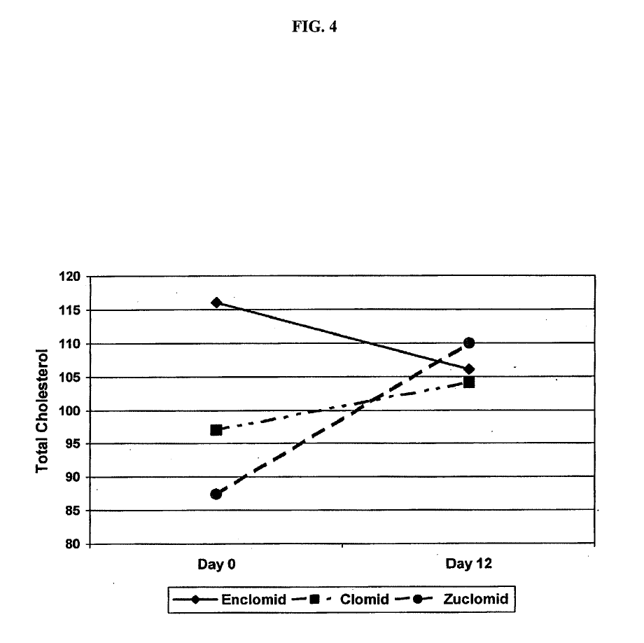

### DETAILED DESCRIPTION

Administration of clomiphene may result in an increase in testosterone levels. As shown in Example 3, administration of a composition comprising trans-clomiphene may increase testosterone levels and may provide an effective therapy for ameliorating or preventing one or more symptoms of low testosterone levels. Surprisingly, the administration of a composition comprising trans-clomiphene to males favorably affects the ratio of DHT to testosterone. As a result, compositions comprising trans-clomiphene may be used to treat benign prostate hypertrophy and prostate cancer in males by lowering the relative ratio of DHT to testosterone. Additionally, administration of a composition comprising trans-clomiphene may be used to lower triglyceride levels.

### 1. Clomiphene Compositions

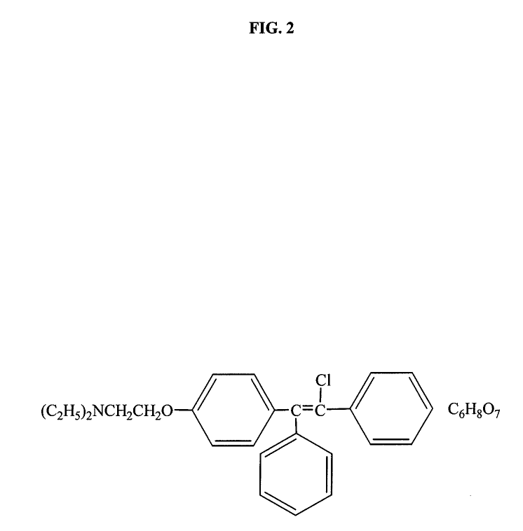

The present invention is related to a composition comprising clomiphene. Clomiphene (FIG. 2) is an antiestrogen related to tamoxifen that blocks the normal estrogen feedback on the hypothalamus and subsequent negative feedback on the pituitary, which may lead to increases in luteinizing hormone (LH) and follicle stimulating hormone (FSH). Clomiphene has been used for therapeutic intervention in men with low testosterone levels. Tenover et al., J. Clin. Endocrinol. Metab. 64:1103, (1987) and Tenover et al., J. Clin. Endocrinol. Metab. 64:1118 (1987) found increased in FSH, LH in both young and old men after treatment with clomiphene. They also found increases in free and total testosterone in men with young men showing significant increases. In men, these increased levels of gonadotropins stimulate the Leydig cells of the testes and result in the production of higher testosterone levels.

Clomiphene is a mixture of two geometric isomers. cis, -Z-, clomiphene (cis-clomiphene or zuclomiphene) and trans-, E-, clomiphene, (trans-clomiphene or enclomiphene). Trans-clomiphene HCI has a melting point of 149° C.-150.5° C., while cis-clomiphene HCI has a melting point of 156.5° C.-158° C. The isomers are also reported to have different in vivo half-life. Clomiphene is currently approved as a mixture of both cis- and trans-isomers, the cis-isomer being present as about 30% to 50% (Merck Manual) for fertility enhancement in the anovulatory patient. When administered to the anovulatory patient, clomiphene improves ovulation by initiating a series of endocrine events culminating in a preovulatory gonadotropin surge and subsequent follicular rupture. The drug is recommended to be administered for 5 days at a dose of up to 100 mg daily. Clomiphene has also been associated with numerous side effects including: blurred vision, abdominal discomfort, gynecomastia, testicular tumors, vasomotor flushes, nausea, and headaches. Furthermore, other studies suggest that clomiphene possesses both genotoxic and tumor enhancement effects. The net outcome of these observations is that clomiphene in its current format, having between 30% and 50% of the cis isomer, would be unacceptable for chronic therapy in men for the treatment of testosterone deficiency.

The trans-isomer of clomiphene is antiestrogenic (AE) while the cis-isomer is the more potent and more estrogenic form and has also been reported to have anti-estrogenic activity. The effect of clomiphene on ovulatory activity is attributed to both forms because the mixture is more effective than trans-clomiphene alone. The trans-isomer aids ovulation at the level of the hypothalamus. The estrogenic isomer cis-clomiphene contributes to enhanced ovulation elsewhere in the physiologic pathway leading to ovulation. The isomers are also reported to have different in vivo half-life. Furthermore, the cis form has been reported to leave residual blood levels for in excess of one month following a single dose.

Vandekerckhove, et al. (Cochrane Database Syst Rev 2000;(2):CD000151 (2000)) noted that ten studies involving 738 men have suggested that anti-estrogens appear to have a beneficial effect on endocrinal outcomes, i.e. testosterone, but there was not enough evidence to evaluate fertility effects. Nevertheless, should clomiphene administration enhance testosterone levels then one could easily conclude that the drug should positively impact the side effects of testosterone deprivation as long as the testes still retain the ability to respond to gonadotropin stimulation.

The clomiphene composition may comprise trans-clomiphene or a pharmaceutically acceptable salt thereof. Cis-clomiphene or a pharmaceutically acceptable salt thereof may also be present in the composition. The ratio of trans-clomiphene to cis-clomiphene may be greater than 1/1 w/w. The ratio of trans-clomiphene to cis-clomiphene may also be greater than 71/29. The composition may comprise about 71, 72, 73, 74, 75, 76, 77, 78, 79, 80, 81, 82, 83, 84, 85, 86, 87, 87, 88, 89, 90, 91, 92, 93, 94, 95, 96, 97, 98, 99 or 100% w/w of active ingredients of trans-clomiphene. Analogs of the trans- and cis-isomers of clomiphene such as those described in Ernst et al., J. Pharmaceut. Sci. 65:148 (1976) may also be used.

a. Formulations

The clomiphene composition may be formulated for oral, intravenous, subcutaneous, buccal, transmucosal, intrathecal, intradermal, intracisternal, or other routes of administration. Suitable pharmaceutical compositions or unit dosage form may be in the form of solids, such as tablets or filled capsules or liquids such as solutions, suspensions, emulsions, elixirs or capsules filled with the same, all for oral use. The composition may also be in the form of sterile injectable solutions or emulsions for parenteral (including subcutaneous) use. The composition may also be in the form of patches, ointments, suppositories, inhalants, or lozenges. Other formulations for administration may also be appropriate and will be understood by one of skill in the art. Such pharmaceutical composition and unit dosage forms thereof may comprise additional pharmaceutically acceptable ingredients such as adjuvants, diluents, carriers, or excipients in conventional proportions.

Pharmaceutical formulations may be in the form of sustained release formulations prepared as described for example in U.S. Pat. No. 6,221,399, Japanese patent 4-312522, Meshali et al, Int. J. Phar. 89:177-181 (1993), Kharenko et al, Intern. Symp. Control Rel. Bioact. Mater. 22:232-233 (1995), WO 95/35093, Dangprasit et al., Drug. Devel. and Incl. Pharm. 21 (20):2323-2337 (1995); U.S. Pat. Nos. 6,143,353, 6,190,591, 6,096,338, 6,129,933, 6,126,969, 6,248,363 and other sustained release formulations well known in the art.

### 2. Methods of Treatment

As used herein, “treatment” or “to treat” or “treating” when referring to protection of a mammal from a condition, means preventing, suppressing, repressing, or eliminating the condition. Preventing the condition involves administering the composition to a mammal prior to onset of the condition. Suppressing the condition involves administering the composition to a mammal after induction of the condition but before its clinical appearance. Repressing the condition involves administering the composition to a mammal after clinical appearance of the condition such that the condition is reduced or maintained. Eliminating the condition involves administering the composition to a mammal after clinical appearance of the condition such that the mammal no longer suffers the condition.

Several studies have been conducted on the effects of clomiphene treatment for conditions other than anovulation described above. For example, studies have been conducted to determine whether or not clomiphene could be used to improve fertility in men by improving semen quality. Homonnai et al. Fertil. and Steril 50:801 (1988) saw increases in sperm concentration and count but others have not. (See e.g., Sokel, et al., Fertil. and Steril. 49:865 (1988); Check, et al., Int. J. Fertil. 34:120 (1989); Purvis, et al., Int. J. Androl 21:109 (1989); and Breznik, Arch. Androl. 21:109 (1993).) One group saw a deterioration in the percentage of normal sperm with long-term treatment. Shamis, et al., Arch. Androl 21:109 (1991). A WHO study showed no changes in semen quality or fertility after 6 months of treatment. (Anonymous Androl. 15:299 (1992).) A meta-analysis seems to confirm that testosterone levels go up in men with poor quality sperm but not fertility. (Vanderkerckhove, et al., 2000). Studies have also suggested that long term treatment with clomiphene does not seem to have a drastic deleterious effect on health, although it did show that treatment resulted in poorer sperm quality after 4 months. Studies have kept men on clomiphene for as long as 18 months and at levels of 25 mg per day or 100 mg every other day.

In 1991, Guay et al (Urology 38:377 (1991)) suggested that clomiphene could treat sexual dysfunction in men. Their hypothesis seems to be that sexual function follows testosterone levels. This was supported by early studies showing positive influence of androgens and sexual function, Davidson, et al., J. Clin. Endocrinol. Metab. 48:955 (1979), and studies that rated sleep-related erections as a strong response to T, Cunningham, et al., J. Clin. Endocrinol. Metab. 70:792 (1990). However, in 1995, Guay et al. (Gray, et al., J. Clin. Endocrinol. Metab. 80:3546 (1995)) published a study in which they saw increase in LH, FSH, and testosterone after 2 months of clomiphene but no effects on erectile dysfunction. There might be some advantage for young men and specific groups of older men, but it seems that just raising the testosterone level is not enough. Effects of testosterone on sleep-related erections may have been taken too seriously (Herskowitz, et al., J. Psychosomat. Res. 42:541 (1997)).

a. Hypogonadism

Hypogonadism may be treated by administering to a patient in need thereof an effective amount of a clomiphene composition. The condition associated with hypogonadism may include, but is not limited to, reduction of muscle mass, limitation of body performance capacity, reduction of bone density, reduction of libido, reduction of potency, benign prostatic hyperplasia, oligospermia or azoospermia, absence or regression of secondary sexual characteristics, reduction of muscle mass, fatigue, and depression.

b. Benign Prostate Hypertrophy (BPH)

BPH may be treated by administering to a patient in need thereof an effective amount of a clomiphene composition. Testosterone levels may be increased by administering the clomiphene composition while positively affecting the ratio of testosterone to DHT compared to testosterone supplements.

c. Prostate Cancer

Prostate cancer may be treated by administering an effective amount of a clomiphene composition. Administration of the compositions may decrease the ratio of testosterone to DHT compared to testosterone supplements. Preferably, in treating prostate cancer, the composition may be administered in a treatment regimen also including a chemotherapeutic. Exemplary chemotherapeutics include Taxotere® (docetaxel), Novantrone® (mitoxantrone hydrochloride), Emcyt® (estramustine sodium phosphate) doxorubicin, ketoconazole and vinblastine although other chemotherapeutics may also be used. In some cases, the chemotherapeutic may be administered closely in time to the dosage of the composition. In other cases the chemotherapeutic may be administered to the patient during the same period of time in which the patient is undergoing treatment with the clomiphene composition. In still other cases, treatment with the clomiphene composition may precede or follow treatment with the chemotherapeutic.

d. Elevated Triglycerides

Elevated triglycerides may be treated by administering an effective amount of a clomiphene composition. The patient may be a male or a female.

e. Methods of Administration

The clomiphene compositions may be administered orally. They may also be administered by the intravenous, subcutaneous, buccal, transmucosal, intrathecal, intradermal, intracisternal or other routes of administration. Depending on the needs of the patient, and the judgment of the prescribing physician, DHT and serum testosterone levels may be measured and dosages may be altered to achieve beneficial levels of DHT and testosterone. Testosterone levels may also be measured for a sufficient increase in the serum testosterone levels to achieve the desired physiological results associated with normal testosterone described above. Likewise, triglyceride levels may be measured before and/or after administration to determine the appropriate dosage.

f. Dosages

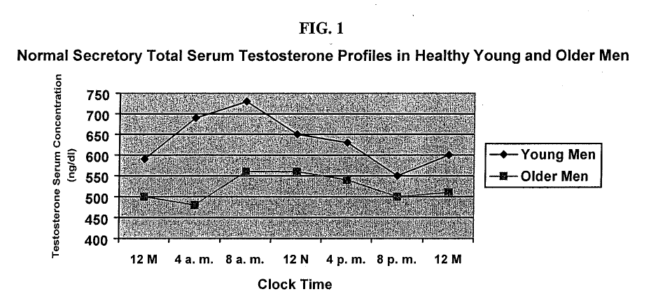

In general, the clomiphene composition may be administered as part of a dosage regimen designed to give rise to serum testosterone levels that mimic or correspond to the normal secretary total serum testosterone profile described in FIG. 1. For example, according to FIG. 1 a dosage of the preferred composition may be administered in a pharmaceutical formulation that would give rise to peak serum testosterone levels between about 4 a.m. and noon. More specifically, the composition could be administered to give rise to peak serum testosterone levels at around 8 a.m. Alternatively, the compositions may be administered to give rise to peak serum testosterone levels at any time of day deemed suitable by the patient and/or the prescribing physician. In another alternative, such as in some of the cases where the patient has elevated triglycerides, but also in other cases depending on the patient's condition and symptoms, the prescribing physician may wish to administer the compositions without regard to serum testosterone levels.

The clomiphene composition may be administered in one or more dosages from about one mg to about 200 mg (although the determination of optimal dosages is with the level of ordinary skill in the art).

The following Examples are meant to be illustrative of the invention and are not intended to limit the scope of the invention as set out in the appended claims.

### EXAMPLE 1

### Effects of Clomids on Serum Testosterone and Cholesterol in Male Baboons

Adult, male, Baboons were given 1.5 mg/kg of Clomid, Enclomid (trans-Clomid) or Zuclomid (cis-Clomid) for 12 consecutive days. The samples analyzed were sera taken on the day of first treatment before being given test article (day 0), after 12 days of treatment (day 12) and 7 days after the last treatment (end or wash-out).

### 1. Effects on Body Weight and Serum LH, FSH, PRL and Testosterone

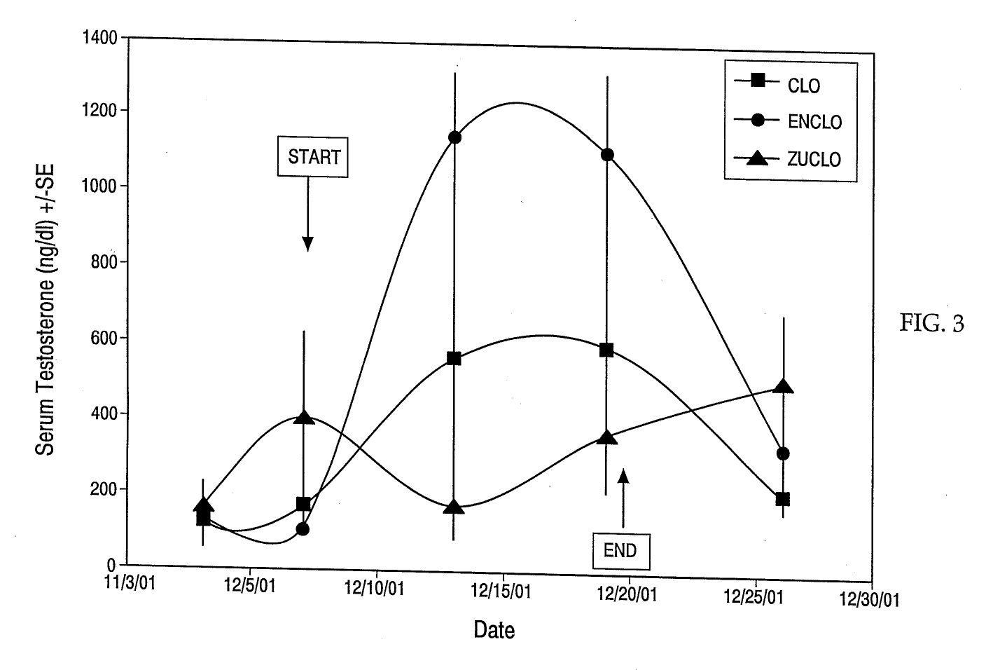

There were significant increases in total serum testosterone in the group receiving Enclomid. See Table 1. There were no differences among groups in the baseline period or at day 0. There were also no differences among the three groups 7 days after treatment (the washout period). However, Enclomid produced higher levels of testosterone compared to Clomid and Zuclomid on day 6 (p=0.03 and p=0.00002 respectively) and compared to Zuclomid on day 12 (p=0.047). Zuclomid clearly did not raise total serum testosterone to any extent. Compared to the animals receiving Enclomid, the animals receiving Clomid exhibited more variable total testosterone levels on day 6 and later as judged by their coefficients of variations. When we looked at the time course of the effects (FIG. 3), we determined that only Enclomid significantly and statistically raised total serum testosterone on days 6 and 12 compared with either baseline or day 0 values. Moreover, cessation of Enclomid treatment, resulted in a significant drop in the level of total serum testosterone between day 12 and day 18 (washout). This indicates that Enclomid is readily cleared from the circulation consistent with the metabolic clearance seen for Enclomid in humans. Enclomid was clearly better and more consistent than Clomid itself and Zuclomid was ineffective.

TABLE I
Serum Testosterone Levels (ng/dl)

| | | baseline | 0 day | 6 days | 12 days | wash-out |
|---|---|---|---|---|---|---|
| Group | ID | Dec. 3, 2001 | Dec. 7, 2001 | Dec. 13, 2001 | Dec. 20, 2001 | Dec. 26, 2001 |
| CLO | 7500 | 79.01 | 76.15 | 940.97 | 891.5 | 150.9 |
| | 9012 | 97.55 | 305.24 | 585.92 | 555.6 | 316.3 |
| | 9097 | 158.06 | 102.94 | 151.12 | 318.9 | 143.6 |
| | Mean | 111.5 | 161.4 | 559.3 | 588.7 | 203.6 |
| | SD | 41.3 | 125.2 | 395.6 | 287.7 | 97.7 |
| ENCLO | 7223 | 64.57 | 74.96 | 1223.8 | 633.6 | 307.2 |
| | 8021 | 166.86 | 133.59 | 1128.2 | 1466 | 399.2 |
| | 8369 | 170.45 | 106.47 | 1081.1 | 1166 | 271 |
| | Mean | 134.0 | 105.0 | 1144.4 | 1088.5 | 325.8 |
| | SD | 60.1 | 29.3 | 72.7 | 421.6 | 66.1 |
| ZUCLO | 7438 | 124.84 | 210.4 | 137.51 | 314.5 | 359.7 |
| | 8292 | 104.66 | 67.37 | 169.98 | 406.1 | 860.5 |
| | 10098 | 282.29 | 904.82 | 227.95 | 353.0 | 274.1 |
| | Mean | 170.6 | 394.2 | 178.5 | 357.9 | 498.1 |
| | SD | 97.3 | 448.0 | 45.8 | 46.0 | 316.8 |
| | ANOVA | p = 0.61 | p = 0.43 | p = 0.007 | p = 0.57 | p = 0.256 |
| | K-W | p = 0.56 | p = 0.84 | p = 0.051 | p = 0.079 | p = 0.252 |

There were no changes in serum LH or FSH. The ratio of total serum testosterone to LH followed the same pattern as total serum testosterone, suggesting a lack of dependence (data not shown). There was also no change in body weight during the 12 day study. There was a decrease in serum prolactin (PRL) during the study in the group receiving Enclomid, suggesting an effect of antiestrogen that has been described in part (Ben-Jonathan and Hnasko, 2001) and expected on the basis of the fact that as men age, testosterone declines and Prolactin increase (Feldman et al., 2002).

2. Effects on Cholesterol levels

Treatment with Enclomid tended to decrease serum cholesterol and Zuclomid tended to increase the same parameter. Preliminary analysis indicated that the changes in cholesterol levels were not statistically significant and that the changes were within the normal range. Due to the observed trend for the two isomers to demonstrate opposite effects on cholesterol levels over a short period of time, further analysis was conducted.

Detailed analysis indicated that Enclomid resulted in an 8% decrease in serum cholesterol levels. Conversely, treatment with Zuclomid resulted in a 22% increase in serum cholesterol levels. Treatment with Clomid resulted in a slight increase in serum cholesterol levels. The opposite effect of Enclomid and Zuclomid on serum cholesterol levels is not unexpected given that the isomers have, alternatively, estrogen agonist or antagonist activity. These results indicate that Enclomid may be used for treating patients with high cholesterol levels. These results also indicate that Enclomid may be more benign than Zuclomid with respect to serum cholesterol if used chronically for increasing testosterone levels.

### 3. Effects on Clinical Chemistry Parameters

The mean values for each parameter did not differ among the three groups for any test parameter at the beginning of the study as determined by ANOVA or by the Kruskal-Wallis test. All groups exhibited normal values at each parameter except for (1) serum sodium; a related calculated parameter, anionic gap, which were low for all nine baboons throughout the trial; (2) serum glucose; and (3) BUN which were high on day 0 for the group which would be treated with Enclomid. On day 12 of treatment and 7 days after treatment (washout), there were no differences among groups for any parameter except anionic gap that showed that the Clomid and Zuclomid groups had lower values than the Enclomid group. The values of serum sodium and anionic gap appear to be anomalies associated with this group of baboons.

There were substantive effects on the red blood cell population with Enclomid and Zuclomid and on hematocrit with Zuclomid. All the compounds lower the mean cell hemoglobin concentration (MCHC) either at day 0 or at the endpoint. With no change in mean cell hemoglobin (MCH) and an increase in the mean cell volume (MCV), the lowering of MCHC is predictable. Although testosterone might be expected to raise hematocrit, only Zuclomid treatment, which did not increase total serum testosterone, demonstrated a statistical difference. Clearly, men in a clinical trial that uses Zuclomid should be monitored for the characteristics of their red blood cell population. Enclomid would be predicted to have less of an effect.

There appears to be a clear effect of 12-day Enclomid treatment on platelets although the values found stayed within the normal range. One thing to consider here is the sexual dimorphism in platelet counts between male and female baboons (279 for males vs. 348 for females). This is likely to be due to hormones. Since the Enclomid group demonstrated increased testosterone, the lowering of the platelet count could be secondary to the change in testosterone in this group. Moreover, treatment with Enclomid pushed the platelet count to its normal male level from a day 0 level that was the high end of the normal range for this group. Enclomid would not necessarily predict a deleterious effect on platelets.

All the Clomids tested had effects on the white blood cell (WBC) population, the most striking was that of Enclomid on raising the counts of lymphocytes and eosinophiles. The effects are not as straightforward as they would seem to be. There appears to be a strong effect of Enclonud on lowering the percent of granulocytes in the blood. The effects are very strong after the 7-day washout period when the values are decreased below the normal range. (This time course could reflect the relatively long time required to affect change the WBC population.) There is little sexual dimorphism in baboons with respect to the white blood cell populations, so the effects are more likely to be due to the compound itself than changes in testosterone. However, when we look at the calculated count of granulocytes using the WBC count, we find no differences in granulocyte count due to any compound. Concomitantly, it is the lymphocyte story that is the most interesting. Both the count and per cent lymphocytes in the population increase with Enclomid treatment. Whereas the mean values of per cent lymphocytes remain in the normal range, given the trend for an increase in WBC count, the net effect is an increase in lymphocyte count with Enclomid. This eosinophil result is analogous. There is a clear implication for treating men who have low lymphocytes, such as men who are HIV-positive. Since Enclomid is unlikely to lower lymphocytes based on this result, a case could be made for its use in the population of men with AIDS. These individuals are often treated with agents that are intended to raise testosterone due to the wasting effects of disease. Low liver and kidney toxicity and favorable effects on cholesterol and lipids are also highly favored attributes for any medication intended for use HIV-positive men who are already compromised by their disease.

The increase in serum glucose with Clomid or Zuclomid was within the normal range. In the case of Enclomid where the mean serum glucose values were high on day 0, there were no increases with treatment. There was no evidence that Enclomid would have a deleterious effect on blood glucose.

No clearly adverse effects on liver function are apparent as judged by the enzymes AST and ALT. The trend in these values was a decrease with treatment. An increase in the level of enzymes in the serum would indicate liver damage. ALT/SGPT was out of range low at the end of the study for the Clomid group although the differences over the treatment period were not statistically significant. The changes with Enclomid and Zuclomid were within the normal range. AST is depressed in pregnancy; thus the action of an estrogen agonist such as Zuclomid in lowering the marginal AST level could be rationalized. Alkaline phosphatase (ALP) is also found in the liver and is elevated various disease states. The lowering of ALP argues further against hepatic damage. There were no changes in serum albumin, also a liver product. A strong suppression of serum albumin over an extended time period could contribute to free serum steroid hormone levels in humans although a more important role is played by sex hormone binding globulin. As a bottom line, none of the compounds could be linked to liver damage on the basis of the parameters assayed.

Osteoblastic activity and diseases of the bone are accompanied by high serum ALP values. ALP was not elevated following Zuclomid treatment and was decreased in value following Enclomid treatment. The trends would predict a more benign result for the use of Enclomid compared to Zuclomid.

Although BUN and BUN/creatinine were altered during the study in the Clomid and Enclomid groups, the lack of a definitive change in creatinine argues against renal dysfunction. A loss of glomerular filtration capacity would result in an increase in BUN. Decreased BUN occurs in humans due to poor nutrition (not likely in a controlled setting), or high fluid intake (presumably accompanied by edema). Also, despite an increase in total serum testosterone between day 0 and Day 12 with Enclomid, there were no differences between serum creatinine values, arguing against an increase in muscle mass over this short time interval.

Serum sodium levels were lower than reference values for all animals throughout the study. Serum carbon dioxide was higher than reference values on day 12 for the Clomid and Zuclomid groups. Serum anion gap was lower for all animals throughout the study, paralleling the sodium results. Enclomid raised this parameter towards normal values. The electrolyte imbalances detected in the test animals throughout all treatment periods remains elusive but might be part of the same fluid derangement phenomenon suggested by the BUN results.

The foregoing results indicate that Enclomid is more effective than Clomid or Zuclomid at enhancing total serum testosterone. Zuclomid is clearly not effective and that deficiency limits any use of Clomid for hypogonadism, particularly since the Zuclomid component of Clomid would predominate in the circulation over time given its longer half-life.

Enclomid appeared to be relatively benign in all aspects when compared to Zuclomid and, often, even Clomid. This is particularly true when consideration is given to the trend of Enclomid to lower cholesterol, and liver enzymes as opposed to Zuclomid's trend to raise the same parameters. The surprising trend for Enclomid to raise the lymphocyte count may be useful for men with AIDS if it can be shown the CD4+ subpopulation of lymphocytes is not lowered or is enhanced.

### EXAMPLE 2

### Method for Increasing Testosterone Level in Men using Traits-Clomiphene and Mixtures of Trans-Clomiphene and Cis-Clomiphene at Ratios Greater than 1

Prior to administration of trans-clomiphene, blood samples are taken from subject males and testosterone levels are measured using methodologies described for example in Matsumoto, et al. Clin. Endocrinol. Metab. 56; 720 (1983) (incorporated herein by reference). Sex hormone binding globulin (SHBG), both free and bound to testosterone, may also be measured as described for example in Tenover et al. J. Clin. Endocrinol. Metab. 65:1118 (1987) which describe measurement of SHBG by both a [³H] dihydrotestosterone saturation analysis and by radioimmunoassay. Non-SHBG-bound testosterone levels (bioavailable testosterone) are also measured for example according to Tenover et al. J. Clin. Endocrinol and Metab. 65:1118 (1987). See also Soderguard et al. J. Steroid Biochem 16:801 (1982) incorporated herein by reference.

Patients are given daily dosages of 1.5 mg/kg clomiphene, wherein the ratio of trans-clomiphene to cis-clomiphene is greater than 1. Patients are monitored for testosterone levels such that the dosage amount and dosage frequency may be adjusted to achieve therapeutic levels of testosterone in the patient.

### EXAMPLE 3

### Method for Decreasing Triglyceride Levels in Men using Trans-Clomiphene and Mixtures of Trans-Clomiphene and Cis-Clomiphene at Ratios Greater than 1

Prior to administration of trans-clomiphene, blood samples are taken from subject males and triglyceride levels are measured using methodologies described for example in Cooper, et al. (Selected methods for the small clinical chemistry laboratory. W. R. Faulkner and S. Meites, eds. Am. Assoc. for Clin. Chem., Washington, D.C. Pages 165-174), which is incorporated herein by reference.

Patients are given daily dosages of 1.5 mg/kg clomiphene, wherein the ratio of trans-clomiphene to cis-clomiphene is greater than 1. The dosage amount and dosage frequency of clomiphene may be adjusted to achieve therapeutic levels of triglycerides in the patient by monitoring for triglyceride levels on different days of the treatment.

### EXAMPLE 4

### Method for Decreasing Triglyceride Levels in Women using Trans-Clomiphene and Mixtures of Trans-Clomiphene and Cis-Clomiphene at Ratios Greater than 1

Prior to administration of trans-clomiphene, blood samples are taken from subject females and triglyceride levels are measured as in Example 3.

Patients are given daily dosages of 1.5 mg/kg clomiphene, wherein the ratio of trans-clomiphene to cis-clomiphene is greater than 1. The dosage amount and dosage frequency of clomiphene may be adjusted to achieve therapeutic levels of triglycerides in the patient by monitoring for triglyceride levels on different days of the treatment.

### EXAMPLE 5

### Comparison of Androxal™ to Androgel®

A placebo controlled challenge study was conducted at the Advanced Biological Research, Inc. (ABR) Clinical Research Center in Hackensack, N.J. to compare orally administered Androxal™ (trans-clomiphene) to Androgel® (testosterone) in hypogonadal men. Androgel® (Solvay Pharmaceuticals, Inc.) consists of a cream that administers exogenous testosterone in a transdermal matrix.

The study enrolled 62 hypogonadal men with testosterone levels less than 300 ng/dl (normal 298-1034 ng/dl) that were randomized into 6 different arms, three doses of Androxal™ (12.5 mg, 25 mg, and 50 mg), placebo, and both high and low doses of Androgel®. Half of the men in each of the Androxal™ and placebo arms were randomized into cohorts that underwent in-clinic sessions on days 1 and 14 to determine pharmacokinetic parameters for Androxal™ as well as cyclical changes in testosterone. The placebo and Androxal™ doses were administered in a double blind fashion. The Androgel® cream was administered in an open label fashion. Half of the Androgel® patients underwent in-clinic sessions similar to the other patients in the study. Following the two week drug exposure patients were followed for an additional seven to ten days to determine the status of their testosterone levels.

There were no side effects noted in either the Androxal™ or Androgel® arms of the study that were different than placebo. Furthermore, all doses produced statistically significant changes in testosterone from baseline testosterone levels. The low, mid and high doses of Androxal™ achieved mean increases of 169, 247, and 294 ng/dl respectively, while those of Androgel® 5G, the lowest approved dose, and Androgel® 10G, the highest approved dose, produced changes from baseline that were 212 and 363 ng/dl. These values were statistically indistinguishable from those changes achieved with Androxal™. This inability to show differences between Androxal™ and Androgel® appears to result from the highly variable results found when Androgel® is used. For example the 50 mg dose of Androxal™ raised mean total testosterone to 589±172 ng/dl after 15 days, a coefficient of variation (CV) of 29% and similar to the placebo group (36%). On the other hand Androgel® 5G and 10G yielded mean total testosterone values 473±289 ng/dl and 608±323 ng/dl, CV's of 61 % and 53% respectively.

After 14 days of Androxal™ therapy all doses were associated with a total testosterone diurnal pattern similar to the placebo group, i.e. a morning peak, a mid-day trough and a rise overnight. Without being bound by theory, this pattern may be due to the mode of action of Androxal™, which appears to be mediated through effects on the hypothalamic-pituitary axis. The diurnal pattern for men on Androgel® was nearly flat. However, spikes in total testosterone for Androgel® were associated with dosing and often exceeded the normal high level of 1,034 ng/dl. Certain individuals on Androgel® 10G were able to achieve peak levels of total testosterone of over 2500 ng/dl.

The effect on serum dihydroxytestosterone (DHT) levels were also measured. Men on Androxal™ experienced a favorable shift in their DHT to total testosterone. For example men on the 50 mg dose of Androxal™ experienced a DHT/TT ratio of 0.83 as compared to the placebo group ratio of 1.07. By contrast the DHT/TT ratio for either of the Androgel® groups was >1.5. The results indicate that men on Androgel® were gaining DHT faster than total testosterone. Thus the normal levels of DHT was disrupted relative to testosterone in men on Androgel® therapy.

Results of clinical chemistry parameters also indicated, unexpectedly, that men on Androxal™ experienced a non-dose dependent reduction in triglycerides. The reduction in triglycerides averaged a decrease of 19.1 % after two weeks of therapy. This compared to a 5.9% reduction for the placebo group and increases of 0.3% and 22% for the Androgel® 5G and 10G respectively.

Based on this study we infer a number of potential advantages for Androxal™ as a potential therapy. First, Androxal™ appears to raise total testosterone into the normal range in a highly consistent manner without abnormally high spikes in serum testosterone. Secondly, Androxal™ appears to improve the DHT/TT ratio, a potentially important consideration from the standpoint of prostate cancer. Thirdly, the maintenance of a normal diurnal pattern improves testosterone levels in a more natural fashion. Finally, the tendency to lower triglycerides may be an advantage to many men.

1. A method for treating a condition selected from the group consisting of benign prostate hypertrophy, prostate cancer, elevated triglycerides, high cholesterol and hypogonadism comprising administering to a patient in need thereof a composition comprising trans-clomiphene or an analog thereof or pharmaceutically acceptable salt or solvate thereof and optionally one or more pharmaceutically acceptable diluents, adjuvants, carriers or excipients.

2. The method of claim 1, wherein the composition further comprises cis-clomiphene or an analog thereof or pharmaceutically acceptable salt or solvate thereof and wherein the ratio of trans-clomiphene to cis-clomiphene is greater than 71/29.

3. The method of claim 1 wherein the composition consists essentially of an effective amount of trans-clomiphene or an analog thereof or a pharmaceutically effective salt or solvate thereof, and optionally one or more pharmaceutically acceptable diluents, adjuvants, carriers, or excipients.

4. The method of claim 1, wherein the condition is selected from the group consisting of benign prostate hypertrophy, prostate cancer, and hypogonadism.

5. The method of claim 4, wherein the composition is formulated for oral administration.

6. The method of claim 4, wherein the composition is formulated for extended release.

7. The method of claim 4, wherein the composition is administered to give rise to peak serum testosterone levels at around 8 a.m.

8. The method of claim 4, wherein the condition is benign prostate hypertrophy.

9. The method of claim 4, wherein the condition is prostate cancer.

10. The method of claim 4, wherein the composition is administered in a dosage regimen further comprising a chemotherapeutic.

11. The method of claim 7 wherein the condition is hypogonadism and the patient suffers from a symptom selected from the group consisting of reduction of muscle mass, limitation of body performance capacity, reduction of bone density, reduction of libido, reduction of potency.

12. The method of claim 4 wherein the patient is a mammal.

13. The method of claim 4 wherein the patient is a human.

14. The method of claim 1 wherein the condition is selected from the group consisting of elevated triglycerides and high cholesterol.

15. The method of claim 12, wherein the patient is male.

16. The method of claim 12, wherein the patient is female.

17. The method of claim 1, wherein the composition is formulated for oral administration.

18. The method of claim 1, wherein the composition is formulated for extended release.
